SEQUENCE LISTING (41 sequences)

SEQ ID NO: 1 - DNA, 46 bp, synthetic construct
  source 1..46: mol_type = other DNA
gaaggtgaccaagttcatgcttgtttctgcatctaaagcgatttgc

SEQ ID NO: 2 - DNA, 46 bp, synthetic construct
  source 1..46: mol_type = other DNA
gaaggtcggagtcaacggatttgtttctgcatctaaagcgatttga

SEQ ID NO: 3 - DNA, 29 bp, synthetic construct
  source 1..29: mol_type = other DNA
attcgctgggaggtacaaaatattacact

SEQ ID NO: 4 - DNA, 46 bp, synthetic construct
  source 1..46: mol_type = other DNA
gaaggtgaccaagttcatgcttaacatgttcatttggttccctttt

SEQ ID NO: 5 - DNA, 46 bp, synthetic construct
  source 1..46: mol_type = other DNA
gaaggtcggagtcaacggatttaacatgttcatttggttccctttc

SEQ ID NO: 6 - DNA, 29 bp, synthetic construct
  source 1..29: mol_type = other DNA
agaactaataaattcaccaaattcgctct

SEQ ID NO: 7 - DNA, 46 bp, synthetic construct
  source 1..46: mol_type = other DNA
gaaggtgaccaagttcatgctccaatataaatcaggtacaacattt

SEQ ID NO: 8 - DNA, 46 bp, synthetic construct
  source 1..46: mol_type = other DNA
gaaggtcggagtcaacggattccaatataaatcaggtacaacatta

SEQ ID NO: 9 - DNA, 29 bp, synthetic construct
  source 1..29: mol_type = other DNA
aagaatattgttaaacaattcctcaatgc

SEQ ID NO: 10 - DNA, 46 bp, synthetic construct
  source 1..46: mol_type = other DNA
gaaggtgaccaagttcatgctatataaatggtcttttaaattgcac

SEQ ID NO: 11 - DNA, 46 bp, synthetic construct
  source 1..46: mol_type = other DNA
gaaggtcggagtcaacggattatataaatggtcttttaaattgcag

SEQ ID NO: 12 - DNA, 29 bp, synthetic construct
  source 1..29: mol_type = other DNA
catcctatatttaacgaattttattatat

SEQ ID NO: 13 - DNA, 46 bp, synthetic construct
  source 1..46: mol_type = other DNA
gaaggtgaccaagttcatgctaaaaaaaacattaataaatgagccg

SEQ ID NO: 14 - DNA, 46 bp, synthetic construct
  source 1..46: mol_type = other DNA
gaaggtcggagtcaacggattaaaaaaaacattaataaatgagcca

SEQ ID NO: 15 - DNA, 29 bp, synthetic construct
  source 1..29: mol_type = other DNA
gacacataccaggaggagtttcaattcgg

SEQ ID NO: 16 - DNA, 46 bp, synthetic construct
  source 1..46: mol_type = other DNA
gaaggtgaccaagttcatgctattcctacaattaatctagcaaaat

SEQ ID NO: 17 - DNA, 46 bp, synthetic construct
  source 1..46: mol_type = other DNA
gaaggtcggagtcaacggattattcctacaattaatctagcaaaac

SEQ ID NO: 18 - DNA, 29 bp, synthetic construct
  source 1..29: mol_type = other DNA
tatgctagttaagcgacttttggagatcg

SEQ ID NO: 19 - DNA, 46 bp, synthetic construct
  source 1..46: mol_type = other DNA
gaaggtgaccaagttcatgcttgctgggcctctatctggaccggga

SEQ ID NO: 20 - DNA, 46 bp, synthetic construct
  source 1..46: mol_type = other DNA
gaaggtcggagtcaacggatttgctgggcctctatctggaccgggg

SEQ ID NO: 21 - DNA, 29 bp, synthetic construct
  source 1..29: mol_type = other DNA
aatcccccccctgcgcatgactgaaagcg

SEQ ID NO: 22 - DNA, 46 bp, synthetic construct
  source 1..46: mol_type = other DNA
gaaggtgaccaagttcatgctattttagtaatttagagcttgattc

SEQ ID NO: 23 - DNA, 46 bp, synthetic construct
  source 1..46: mol_type = other DNA
gaaggtcggagtcaacggattattttagtaatttagagcttgattt

SEQ ID NO: 24 - DNA, 29 bp, synthetic construct
  source 1..29: mol_type = other DNA
atagtttaaggtacaattttccatcccag

SEQ ID NO: 25 - DNA, 46 bp, synthetic construct
  source 1..46: mol_type = other DNA
gaaggtgaccaagttcatgctaccacccctccagagagtcagaaaa

SEQ ID NO: 26 - DNA, 46 bp, synthetic construct
  source 1..46: mol_type = other DNA
gaaggtcggagtcaacggattaccacccctccagagagtcagaaag

SEQ ID NO: 27 - DNA, 29 bp, synthetic construct
  source 1..29: mol_type = other DNA
gcccacctaatatactacctccctttggc

SEQ ID NO: 28 - DNA, 46 bp, synthetic construct
  source 1..46: mol_type = other DNA
gaaggtgaccaagttcatgctagactcgttcaaatgacctaaagcc

SEQ ID NO: 29 - DNA, 46 bp, synthetic construct
  source 1..46: mol_type = other DNA
gaaggtcggagtcaacggattagactcgttcaaatgacctaaagct

SEQ ID NO: 30 - DNA, 29 bp, synthetic construct
  source 1..29: mol_type = other DNA
ccaagtatgtacaaatcgaacaatgtgcg

SEQ ID NO: 31 - DNA, 46 bp, synthetic construct
  source 1..46: mol_type = other DNA
gaaggtgaccaagttcatgctggttcattttagcaagagatgaaac

SEQ ID NO: 32 - DNA, 46 bp, synthetic construct
  source 1..46: mol_type = other DNA
gaaggtcggagtcaacggattggttcattttagcaagagatgaaat

SEQ ID NO: 33 - DNA, 29 bp, synthetic construct
  source 1..29: mol_type = other DNA
ctgaacttttccaaccctgcatgatctat

SEQ ID NO: 34 - DNA, 46 bp, synthetic construct
  source 1..46: mol_type = other DNA
gaaggtgaccaagttcatgcttgctgatcagaaaaggtaaagaaaa

SEQ ID NO: 35 - DNA, 46 bp, synthetic construct
  source 1..46: mol_type = other DNA
gaaggtcggagtcaacggatttgctgatcagaaaaggtaaagaaag

SEQ ID NO: 36 - DNA, 30 bp, synthetic construct
  source 1..30: mol_type = other DNA
atatccaaaaattaggctatttacaagaat

SEQ ID NO: 37 - DNA, 46 bp, synthetic construct
  source 1..46: mol_type = other DNA
gaaggtgaccaagttcatgcttgcattgggaacagaggtctgcttc

SEQ ID NO: 38 - DNA, 46 bp, synthetic construct
  source 1..46: mol_type = other DNA
gaaggtcggagtcaacggatttgcattgggaacagaggtctgcttt

SEQ ID NO: 39 - DNA, 30 bp, synthetic construct
  source 1..30: mol_type = other DNA
tgtcttcaggaactttaaaatgtaagggag

SEQ ID NO: 40 - DNA, 21 bp, synthetic construct
  source 1..21: mol_type = other DNA
gaaggtgaccaagttcatgct

SEQ ID NO: 41 - DNA, 21 bp, synthetic construct
  source 1..21: mol_type = other DNA
gaaggtcggagtcaacggatt

(19) United States
(12) Patent Application Publication
(10) Pub. No.: US 2026/0201481 A1
(43) Pub. Date: Jul. 16, 2026

(54) Kompetitive allele-specific PCR (KASP)-based Artemia core molecular marker combination and use thereof

(71) Applicants: Tianjin University of Science and Technology, TIANJIN (CN); Bohai Seafoods Co., Ltd., Shangdong Province (CN)

(72) Inventors: Xuekai HAN, TIANJIN (CN); Xinhua WU, TIANJIN (CN); Ke LI, TIANJIN (CN); Liying SUI, TIANJIN (CN); Bo ZHANG, TIANJIN (CN); Ziying LI, TIANJIN (CN); Hongsheng MA, TIANJIN (CN); Rongsong SUN, TIANJIN (CN)

(21) Appl. No.: 19/005,139

(22) Filed: Dec. 30, 2024

(30) Foreign Application Priority Data
Jan. 2, 2024 (CN) 202410014639.9

(51) Int. Cl. C12Q 1/6888

(52) U.S. Cl. CPC C12Q 1/6888; C12Q 2600/156

(57) ABSTRACT

Disclosed is a kompetitive allele-specific PCR (KASP)-based Artemia core molecular marker combination and use thereof, which relates to the technical fields of molecular biology, plant germplasm resources, and molecular breeding. In the present application, 13 Artemia core markers are screened and designed from 530,000 SNP markers generated by reduced-representation genome sequencing of 290 Artemia resources. The marker combination is used to construct an Artemia SNP fingerprint, detect authenticity of Artemia species, ensure reliability of Artemia resource collection, analyze a genetic relationship of Artemia resources, and guide Artemia breeding.

### CROSS REFERENCE TO RELATED APPLICATION

This patent application claims the benefit and priority of Chinese Patent Application No. 202410014639.9 filed with the China National Intellectual Property Administration on Jan. 2, 2024, the disclosure of which is incorporated by reference herein in its entirety as part of the present application.

### REFERENCE TO SEQUENCE LISTING

A computer readable XML file entitled “Sequence Listing”, that was created on Mar. 7, 2025, with a file size of 37,759 bytes, contains the sequence listing for this application, has been filed with this application, and is hereby incorporated by reference in its entirety.

### TECHNICAL FIELD

The present disclosure relates to the technical fields of molecular biology, plant germplasm resources, and molecular breeding, in particular to a kompetitive allele-specific PCR (KASP)-based Artemia core molecular marker combination and use thereof.

### BACKGROUND

The brine shrimp (Artemia) is a small crustacean, which is widely distributed in inland salt lakes and coastal salt pans around the world. Artemia larvae are easy to hatch, rich in protein and unsaturated fatty acids, and serves as an irreplaceable biological bait for aquatic breeds (especially for larvae of marine fish, shrimp and crabs), which can promote the metamorphosis and disease resistance of aquatic economic animals in the early development stage. In recent years, with the continuous rise in global aquaculture production, the demand for Artemia resources is increasing day by day, and the industry prospects are broad. Although the research on Artemia in China started late, diverse Artemia egg resources all over the world, especially from the major producing areas, have been collected and preserved. However, due to the high similarity in the appearance of Artemia eggs, it is not conducive to the management and utilization of Artemia resources, and the specific genetic background of Artemia is not completely clear, failing to carry out routine breeding and molecular breeding of Artemia quickly and effectively. Therefore, constructing fingerprints for these resources and assigning unique identity information to each resource is of great significance for the collection and identification of Artemia resources and the molecular species certification.

At present, the second-generation molecular markers are still used for the construction of the fingerprint maps of Artemia germplasm resources. With the rapid development of molecular marker technology and next-generation sequencing (NGS) technology, single nucleotide polymorphism (SNP) markers and genomics theories and methods have been deeply applied to the diversity detection and fingerprint construction of germplasm resources. Because SNP markers, with advantages such as whole genome coverage, high throughput, site specificity, codominant inheritance, low error rate, low development and detection costs, will become an important marker type for detection of high-throughput germplasm resources in the future. In recent years, SNP markers have been widely used in fingerprint construction and phylogenetic analysis of staple crops such as wheat, rice, and corn. However, there is no report on SNP molecular markers of KASP technology for the identification of Artemia resources and the species certification.

### SUMMARY

To solve the above technical problems, the present disclosure provides a KASP-based Artemia core molecular marker combination and use thereof. In the present disclosure, 13 Artemia core markers are screened and designed from 530,000 SNP markers generated by reduced-representation genome sequencing of 290 Artemia resources. Construction of SNP fingerprints of Artemia by using the SNP markers provided by the present disclosure can systematically identify the existing Artemia resources in major producing areas worldwide, and establish intellectual property protection mechanism of Artemia species. The SNP markers provided by the present disclosure are used to screen and identify newly collected Artemia resources, certify newly cultivated Artemia species, and carry out Artemia breeding.

To achieve the above objectives, the present disclosure adopts the following technical solutions:

The present disclosure provides a KASP-based Artemia core molecular marker combination, including 13 SNP molecular markers; the 13 SNP molecular markers are Artemia SNP01, Artemia SNP02, Artemia SNP03, Artemia SNP04, Artemia SNP05, Artemia SNP06, Artemia SNP07, Artemia SNP08, Artemia SNP09, Artemia SNP10, Artemia SNP11, Artemia SNP12, and Artemia SNP13, respectively;
the Artemia SNP01 is located on contig14 of Artemia, at a physical position of 585775, with C as a reference base, and A as a substitution base;
the Artemia SNP02 is located on contig30 of Artemia, at a physical position of 3557862, with T as a reference base, and C as a substitution base;
the Artemia SNP03 is located on contig71 of Artemia, at a physical position of 1303074, with A as a reference base, and T as a substitution base;
the Artemia SNP04 is located on contig96 of Artemia, at a physical position of 4160684, with C as a reference base, and G as a substitution base;
the Artemia SNP05 is located on contig210 of Artemia, at a physical position of 3460211, with G as a reference base, and A as a substitution base;
the Artemia SNP06 is located on contig316 of Artemia, at a physical position of 451626, with T as a reference base, and C as a substitution base;
the Artemia SNP07 is located on contig354 of Artemia, at a physical position of 393026, with A as a reference base, and G as a substitution base;
the Artemia SNP08 is located on contig498 of Artemia, at a physical position of 463045, with C as a reference base, and T as a substitution base;
the Artemia SNP09 is located on contig511 of Artemia, at a physical position of 1564272, with A as a reference base, and G as a substitution base;
the Artemia SNP10 is located on contig676 of Artemia, at a physical position of 177489, with C as a reference base, and T as a substitution base;
the Artemia SNP11 is located on contig779 of Artemia, at a physical position of 1201204, with C as a reference base, and T as a substitution base;
the Artemia SNP12 is located on contig2191 of Artemia, at a physical position of 107679, with A as a reference base, and G as a substitution base;
the Artemia SNP13 is located on contig2877 of Artemia, at a physical position of 71092, with C as a reference base, and T as a substitution base; and
the physical positions of the 13 SNP molecular markers are determined by using a contig sequence mounted on a chromosome of Artemia parthenogenetica as a reference genome.

Further, the nucleotide sequences of KASP primers for amplifying the Artemia SNP01 are set forth in SEQ ID NOs: 1 to 3;
the nucleotide sequences of KASP primers for amplifying the Artemia SNP02 are set forth in SEQ ID NOs: 4 to 6;
the nucleotide sequences of KASP primers for amplifying the Artemia SNP03 are set forth in SEQ ID NOs: 7 to 9;
the nucleotide sequences of KASP primers for amplifying the Artemia SNP04 are set forth in SEQ ID NOs: 10 to 12;
the nucleotide sequences of KASP primers for amplifying the Artemia SNP05 are set forth in SEQ ID NOs: 13 to 15;
the nucleotide sequences of KASP primers for amplifying the Artemia SNP06 are set forth in SEQ ID NOs: 16 to 18;
the nucleotide sequences of KASP primers for amplifying the Artemia SNP07 are set forth in SEQ ID NOs: 19 to 21;
the nucleotide sequences of KASP primers for amplifying the Artemia SNP08 are set forth in SEQ ID NOs: 22 to 24;
the nucleotide sequences of KASP primers for amplifying the Artemia SNP09 are set forth in SEQ ID NOs: 25 to 27;
the nucleotide sequences of KASP primers for amplifying the Artemia SNP010 are set forth in SEQ ID NOs: 28 to 30;
the nucleotide sequences of KASP primers for amplifying the Artemia SNP011 are set forth in SEQ ID NOs: 31 to 33;
the nucleotide sequences of KASP primers for amplifying the Artemia SNP012 are set forth in SEQ ID NOs: 34 to 36; and
the nucleotide sequences of KASP primers for amplifying the Artemia SNP013 are set forth in SEQ ID NOs: 37 to 39.

The present disclosure provides a set of KASP primers. The set of KASP primers are KASP primers for amplifying the Artemia core molecular marker combination.

The present disclosure further provides a chip, containing the KASP primers.

The present disclosure further provides an assay kit, including the KASP primers.

The present disclosure further provides use of the Artemia core molecular marker combination, the KASP primers, the chip, or the assay kit in constructing an Artemia SNP fingerprint.

In the present disclosure, genomic DNA of an Artemia to be tested is extracted, and the genomic DNA is used as a template for amplification by the KASP primers to obtain genotype data. The Artemia SNP fingerprint and a database are constructed according to the genotype data. The amplification is programmed as follows: pre-denaturation at 94° C. for 15 min; a first amplification step consisting of 10 cycles of denaturation at 94° C. for 20 s and extension at 61-55° C. for 60 s; and a second amplification step consisting of 26 cycles of denaturation at 94° C. for 20 s and extension at 55° C. for 60 s. A system of the amplification includes: 1 μL of DNA template, 5 μL of 2×KASP Master mix, 0.14 μL of KBD Assay mix, 3.86 μL of ultrapure water, with a total reaction system of 10 μL.

The present disclosure further provides use of the Artemia core molecular marker combination, the KASP primers, the chip, or the assay kit in detecting an Artemia resource.

The present disclosure further provides use of the Artemia core molecular marker combination, the KASP primers, the chip, or the assay kit in classification of Artemia groups.

The present disclosure further provides a method for detecting an Artemia resource, including the following steps:
(1) extracting genomic DNA of an Artemia to be tested, and detecting the genomic DNA by using the Artemia core molecular marker combination, to obtain genotype data of the Artemia to be tested; and
(2) aligning the genotype data of the Artemia to be tested with genotype data of existing Artemia, where the Artemia to be tested is identified as a new resource when there are 3 or more differential loci.

Compared with the prior art, the present disclosure has the following beneficial effects:
In the present disclosure, 13 Artemia core markers are screened and designed from 530,000 SNP markers generated by reduced-representation genome sequencing of 290 Artemia resources. The marker combination can be used to construct an Artemia SNP fingerprint, detect authenticity of Artemia species, ensure reliability of Artemia resource collection, analyze a genetic relationship of Artemia resources, and guide Artemia breeding. The screened 13 core SNP markers have the advantages of high genetic diversity, excellent stability and repeatability, and excellent accessibility and promotional value.

### BRIEF DESCRIPTION OF THE DRAWINGS

FIGS. 1A-1D show SNP fingerprints of 127 Artemia samples in Example 2 of the present disclosure;

FIGS. 2A-2D are clustering diagrams of 127 Artemia resources in Example 2 of the present disclosure;

FIGS. 3A-3D show specific fingerprints of 141 Artemia samples in Example 3 of the present disclosure; and

FIGS. 4A-4D are clustering diagrams of 141 Artemia samples in Example 3 of the present disclosure.

### DETAILED DESCRIPTION OF THE EMBODIMENTS

The following examples are intended to illustrate the present disclosure, but not to limit the scope of the present disclosure. Modifications or substitutions made to methods, steps or conditions of the present disclosure without departing from the spirit and essence of the present disclosure fall within the scope of the present disclosure. All reagents and instruments used in the following examples may be commercially available. Unless otherwise specified, the methods used in the examples are consistent with those commonly used.

The technical solution of the present disclosure will be further described in detail below with reference to examples.

### Example 1 Screening of Artemia Core SNP Markers

In the early stage, 290 Artemia resources were subjected to reduced-representation genome sequencing, specifically Specific-locus amplified fragment (SLAF) sequencing technology. SLAF refers to genotyping by sequencing, by which SNP molecular markers are constructed by selecting appropriate restriction endonucleases combined with high-throughput population sequencing. Firstly, the genomic DNA was digested by restriction endonucleases, and then ligated to the Dual-index adapter. The samples were mixed to construct a small fragment library (364-414 bp) for paired-end sequencing.

### 1. Electronic Enzyme Digestion Evaluation of Genome

In order to check the enzyme digestion efficiency and analyze all markers, the genome was digested with the reference genome according to the loci of the restriction endonucleases, and the corresponding situation was statistically counted for later evaluation.

### 2. SNP Marker Development

Using the Artemia parthenogenetica genome as the reference genome, sequencing reads were aligned to the reference genome using bwa, and SNPs were developed using GATK (v3.8) and samtools (v1.9) methods. The intersection set of SNP markers obtained by these two methods was used as the final reliable SNP marker dataset. A total of 4,644,895 population SNPs were obtained.

### 3. Pre-Screening of Artemia Core Markers

Based on the developed population SNPs, 529,667 highly consistent SNPs were obtained by filtering according to minor allele frequency (MAF: 0.05) and locus integrity (INT: 0.5).

### 4. Acquisition of Artemia Core SNP Markers

(1) The markers were evenly distributed on the genome;

(2) the markers had no deletion loci, namely, the locus integrity was 100%;

(3) the loci with minor allele frequency (MAF) less than 20% were discarded;

(4) the loci with polymorphism information content (PIC) less than 0.35 were discarded;

(5) the loci with p-value greater than 0.01 were preserved by Hardy-Weinberg equilibrium; and

(6) there was no other locus mutation at 100 bp before and after the screened marker.

The markers were screened based on the above conditions, and 50 variable loci were finally determined as candidate markers. KASP primers were designed for 50 candidate SNP markers for genotyping. Finally, 13 KASP markers were selected as SNP markers for subsequent Artemia fingerprinting, as shown in the 13 Artemia core markers listed in Table 1.

TABLE 1
Thirteen (13) Artemia core markers

| | | | | | Substi- |
|---|---|---|---|---|---|
| | | | Physical | Reference | tution |
| NO | Name of SNP | Conting | position | base | base |
| 1 | Artemia SNP01 | Conting14 | 585775 | C | A |
| 2 | Artemia SNP02 | Conting30 | 3557862 | T | C |
| 3 | Artemia SNP03 | Conting71 | 1303074 | A | T |
| 4 | Artemia SNP04 | Conting96 | 4160684 | C | G |
| 5 | Artemia SNP05 | Conting210 | 3460211 | G | A |
| 6 | Artemia SNP06 | Conting316 | 451626 | T | C |
| 7 | Artemia SNP07 | Conting354 | 393026 | A | G |
| 8 | Artemia SNP08 | Conting498 | 463045 | C | T |
| 9 | Artemia SNP09 | Conting511 | 1564272 | A | G |
| 10 | Artemia SNP010 | Conting676 | 177489 | C | T |
| 11 | Artemia SNP011 | Conting779 | 1201204 | C | T |
| 12 | Artemia SNP012 | Conting2191 | 107679 | A | G |
| 13 | Artemia SNP013 | Conting2877 | 71092 | C | T |

### Example 2 Identification of 290 Artemia Resources and Construction of a Fingerprint Using 13 Artemia Core SNP Markers

A total of 290 Artemia germplasm resources from the major producing areas around the world were collected and preserved by the Asian Regional Artemia Reference Center. These resources have only undergone phenotypic identification, and it was impossible to identify whether they were the same Artemia resources. The genetic relationships of the above resources were identified using the 13 Artemia core SNP markers and the KASP primers provided by the present disclosure. The specific experimental steps were as follows:
(1) Preparation of DNA template: a total of 290 samples of Artemia were sampled when they were cultured to adults. Genomic DNAs were extracted by using assay kit method.
(2) Design and synthesis of KASP primers: when designing KASP primers, the selected SNP loci required the addition of FAM-tagged sequence 5′-GAGGTGACCAAGTTCATGCT-3′ (SEQ ID NO: 40) at the 5′-end of the upstream primer-Primer_AlleleX, and HEX-tagged sequence 5′-GAGGTCGGAGTCAACGGATT-3′ (SEQ ID NO: 41) at the 5′-end of the upstream primer-Primer_AlleleY. The primers were synthesized by Beijing Liuhe BGI Co., Ltd. Details of primers are shown in Table 2.

TABLE 2
KASP primer sequences

| | Upstream primer- | Upstream primer- | |
|---|---|---|---|
| Name of SNP | Primer_AlleleX | Primer_AlleleY | Reverse primer |
| Artemia SNP01 | SEQ ID NO: 1 | SEQ ID NO: 2 | SEQ ID NO: 3 |
| Artemia SNP02 | SEQ ID NO: 4 | SEQ ID NO: 5 | SEQ ID NO: 6 |
| Artemia SNP03 | SEQ ID NO: 7 | SEQ ID NO: 8 | SEQ ID NO: 9 |
| Artemia SNP04 | SEQ ID NO: 10 | SEQ ID NO: 11 | SEQ ID NO: 12 |
| Artemia SNP05 | SEQ ID NO: 13 | SEQ ID NO: 14 | SEQ ID NO: 15 |
| Artemia SNP06 | SEQ ID NO: 16 | SEQ ID NO: 17 | SEQ ID NO: 18 |
| Artemia SNP07 | SEQ ID NO: 19 | SEQ ID NO: 20 | SEQ ID NO: 21 |
| Artemia SNP08 | SEQ ID NO: 22 | SEQ ID NO: 23 | SEQ ID NO: 24 |
| Artemia SNP09 | SEQ ID NO: 25 | SEQ ID NO: 26 | SEQ ID NO: 27 |
| Artemia SNP010 | SEQ ID NO: 28 | SEQ ID NO: 29 | SEQ ID NO: 30 |
| Artemia SNP011 | SEQ ID NO: 31 | SEQ ID NO: 32 | SEQ ID NO: 33 |
| Artemia SNP012 | SEQ ID NO: 34 | SEQ ID NO: 35 | SEQ ID NO: 36 |
| Artemia SNP013 | SEQ ID NO: 37 | SEQ ID NO: 38 | SEQ ID NO: 39 |

SEQ ID NO: 1:
GAAGGTGACCAAGTTCATGCTTGTTTCTGCATCTAAAGCGATTTGC;
SEQ ID NO: 2:
GAAGGTCGGAGTCAACGGATTTGTTTCTGCATCTAAAGCGATTTGA;
SEQ ID NO: 2:
GAAGGTCGGAGTCAACGGATTTGTTTCTGCATCTAAAGCGATTTGA;
SEQ ID NO: 3:
ATTCGCTGGGAGGTACAAAATATTACACT.
SEQ ID NO: 4:
GAAGGTGACCAAGTTCATGCTTAACATGTTCATTTGGTTCCCTTTT;
SEQ ID NO: 5:
GAAGGTCGGAGTCAACGGATTTAACATGTTCATTTGGTTCCCTTTC;
SEQ ID NO: 6:
AGAACTAATAAATTCACCAAATTCGCTCT.
SEQ ID NO: 7:
GAAGGTGACCAAGTTCATGCTCCAATATAAATCAGGTACAACATTT;
SEQ ID NO: 8:
GAAGGTCGGAGTCAACGGATTCCAATATAAATCAGGTACAACATTA;
SEQ ID NO: 9:
AAGAATATTGTTAAACAATTCCTCAATGC.
SEQ ID NO: 10:
GAAGGTGACCAAGTTCATGCTATATAAATGGTCTTTTAAATTGCAC;
SEQ ID NO: 11:
GAAGGTCGGAGTCAACGGATTATATAAATGGTCTTTTAAATTGCAG;
SEQ ID NO: 12:
CATCCTATATTTAACGAATTTTATTATAT.
SEQ ID NO: 13:
GAAGGTGACCAAGTTCATGCTAAAAAAAACATTAATAAATGAGCCG;
SEQ ID NO: 14:
GAAGGTCGGAGTCAACGGATTAAAAAAAACATTAATAAATGAGCCA;
SEQ ID NO: 15:
GACACATACCAGGAGGAGTTTCAATTCGG.
SEQ ID NO: 16:
GAAGGTGACCAAGTTCATGCTATTCCTACAATTAATCTAGCAAAAT;
SEQ ID NO: 17:
GAAGGTCGGAGTCAACGGATTATTCCTACAATTAATCTAGCAAAAC;
SEQ ID NO: 18:
TATGCTAGTTAAGCGACTTTTGGAGATCG.
SEQ ID NO: 19:
GAAGGTGACCAAGTTCATGCTTGCTGGGCCTCTATCTGGACCGGGA;
SEQ ID NO: 20:
GAAGGTCGGAGTCAACGGATTTGCTGGGCCTCTATCTGGACCGGGG;
SEQ ID NO: 21:
AATCCCCCCCCTGCGCATGACTGAAAGCG.
SEQ ID NO: 22:
GAAGGTGACCAAGTTCATGCTATTTTAGTAATTTAGAGCTTGATTC;
SEQ ID NO: 23:
GAAGGTCGGAGTCAACGGATTATTTTAGTAATTTAGAGCTTGATTT;
SEQ ID NO: 24:
ATAGTTTAAGGTACAATTTTCCATCCCAG.
SEQ ID NO: 25:
GAAGGTGACCAAGTTCATGCTACCACCCCTCCAGAGAGTCAGAAAA;
SEQ ID NO: 26:
GAAGGTCGGAGTCAACGGATTACCACCCCTCCAGAGAGTCAGAAAG;
SEQ ID NO: 27:
GCCCACCTAATATACTACCTCCCTTTGGC.
SEQ ID NO: 28:
GAAGGTGACCAAGTTCATGCTAGACTCGTTCAAATGACCTAAAGCC;
SEQ ID NO: 29:
GAAGGTCGGAGTCAACGGATTAGACTCGTTCAAATGACCTAAAGCT;
SEQ ID NO: 30:
CCAAGTATGTACAAATCGAACAATGTGCG.
SEQ ID NO: 31:
GAAGGTGACCAAGTTCATGCTGGTTCATTTTAGCAAGAGATGAAAC;
SEQ ID NO: 32:
GAAGGTCGGAGTCAACGGATTGGTTCATTTTAGCAAGAGATGAAAT;
SEQ ID NO: 33:
CTGAACTTTTCCAACCCTGCATGATCTAT.
SEQ ID NO: 34:
GAAGGTGACCAAGTTCATGCTTGCTGATCAGAAAAGGTAAAGAAAA;
SEQ ID NO: 35:
GAAGGTCGGAGTCAACGGATTTGCTGATCAGAAAAGGTAAAGAAAG;
SEQ ID NO: 36:
ATATCCAAAAATTAGGCTATTTACAAGAAT.
SEQ ID NO: 37:
GAAGGTGACCAAGTTCATGCTTGCATTGGGAACAGAGGTCTGCTTC;
SEQ ID NO: 38:
GAAGGTCGGAGTCAACGGATTTGCATTGGGAACAGAGGTCTGCTTT;
SEQ ID NO: 39:
TGTCTTCAGGAACTTTAAAATGTAAGGGAG.

(3) The reaction system is shown in Table 3.

TABLE 3
Reaction mixture system

| Composition | Volume | |
|---|---|---|
| DNA | 1 | μL |
| 2 × KASP Master mix | 5 | μL |
| KBD Assay mix | 0.14 | μL |
| Water | 3.86 | μL |
| Total volume | 10 | μL |

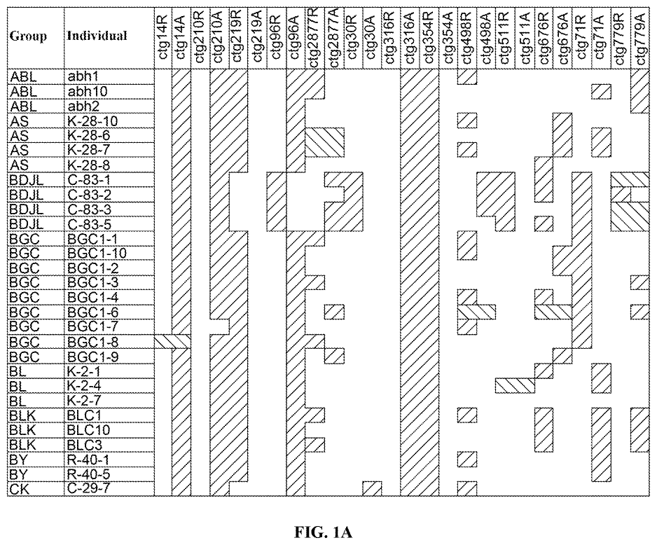

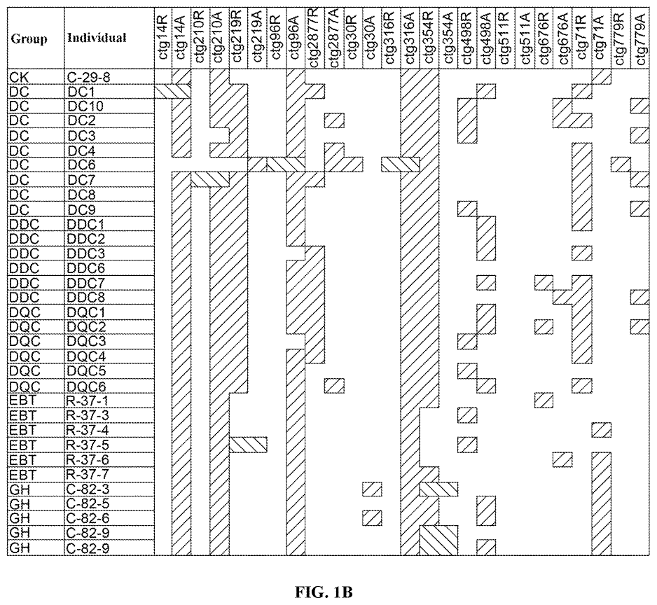

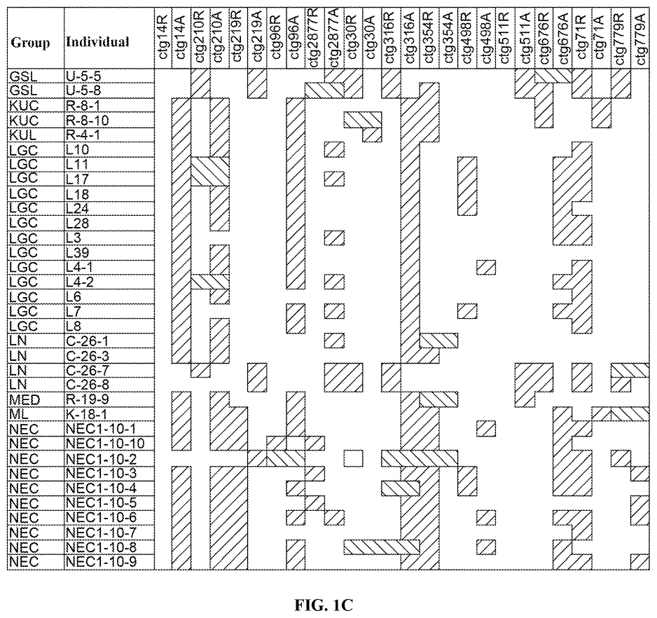

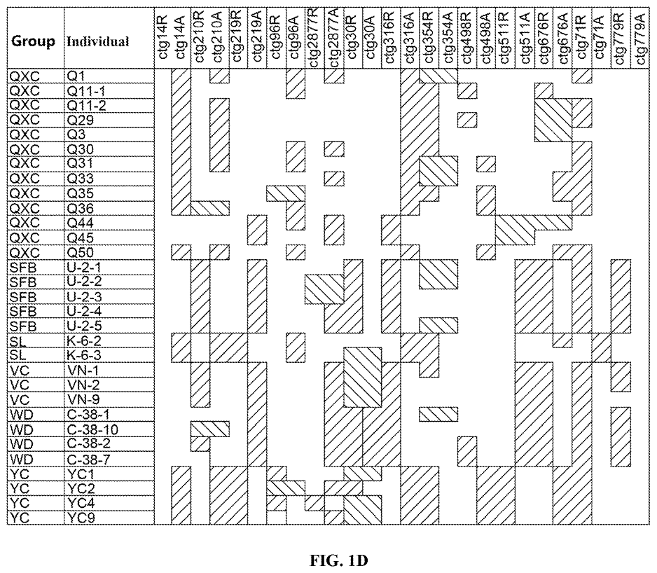

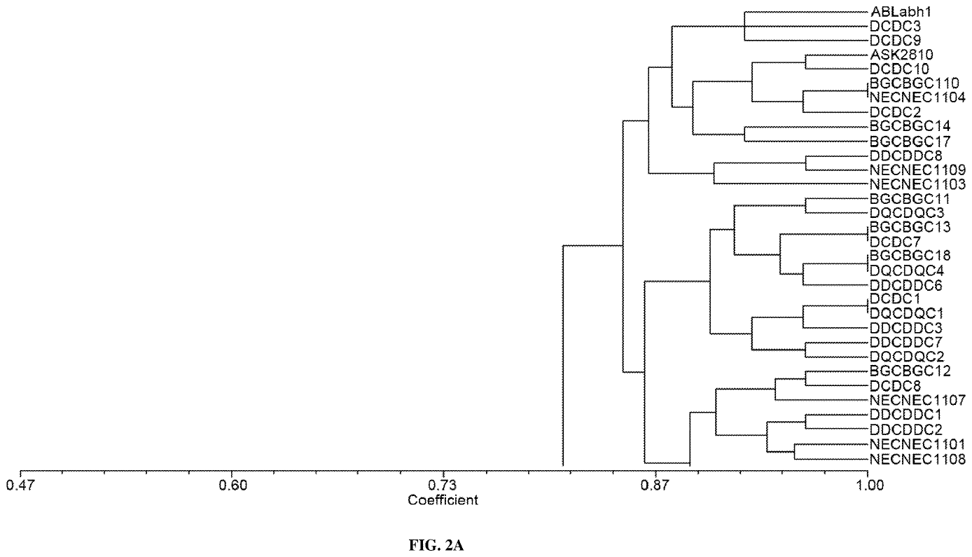

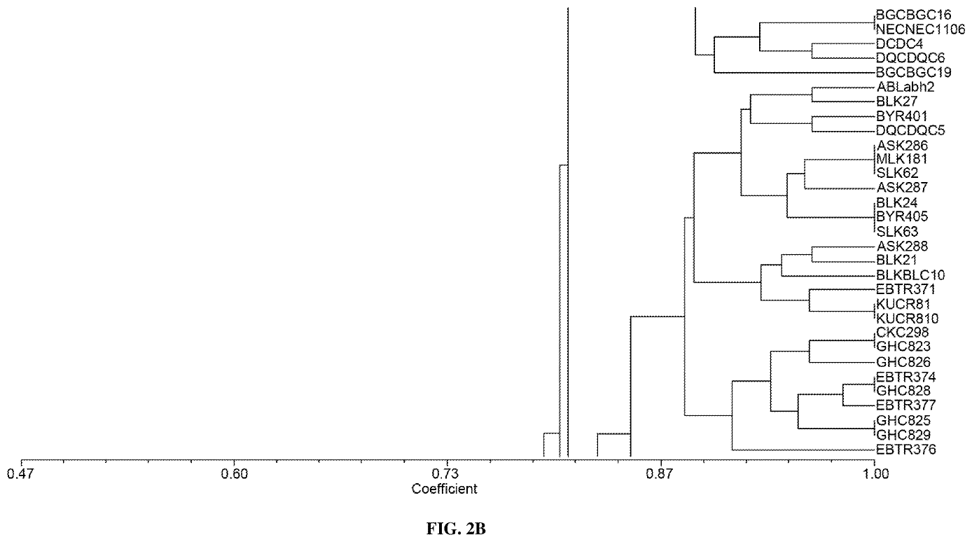

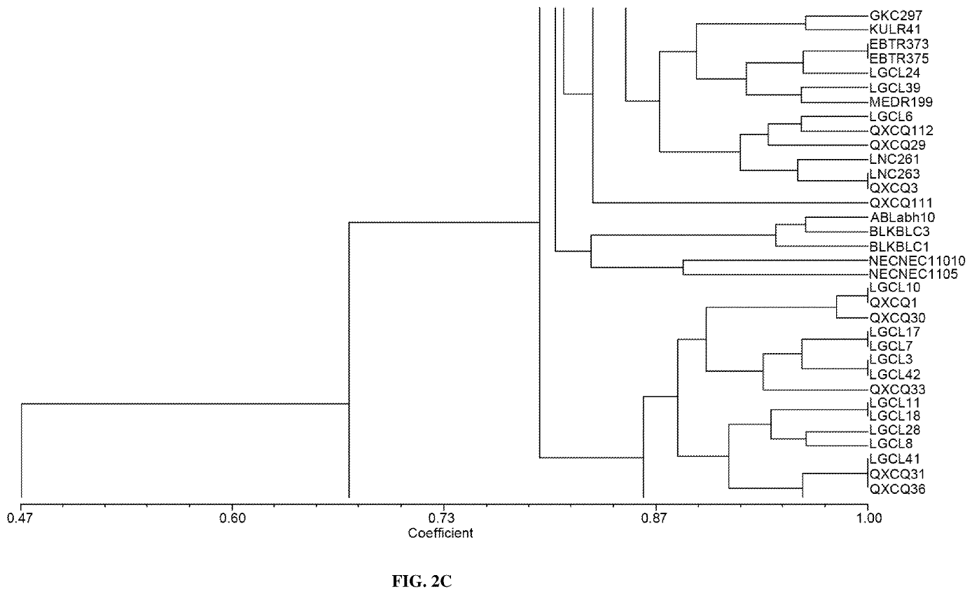

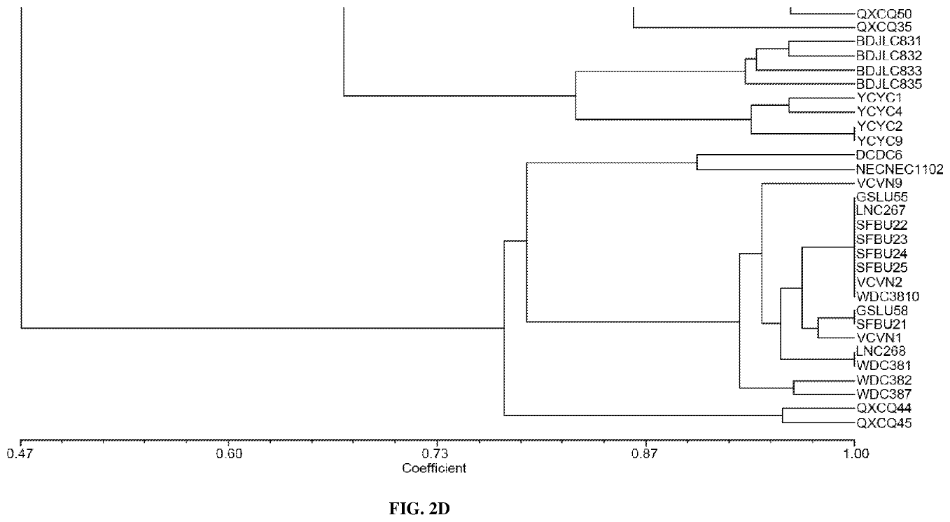

(4) The reaction conditions were as follows: pre-denaturation at 94° C. for 15 min; the first amplification step consisting of 10 cycles of denaturation at 94° C. for 20 s and extension at 61-55° C. for 60 s; and the second amplification step consisting of 26 cycles of denaturation at 94° C. for 20 s and extension at 55° C. for 60 s.
(5) Result analysis: Data pre-processing and analysis were conducted by karaken software. Genotyping data were exported in the form of Excel files. The bands with the same genotype as the reference genome were marked as ‘1’, those with different genotypes were marked as ‘0’, and the deletions were marked as ‘-’. The SNP fingerprints of 290 samples were constructed by using 13 high-quality KASP markers. After eliminating samples with consistent genotypes, the specific fingerprints of 127 samples were finally obtained (FIGS. 1A-1D). The sequence matrix was constructed. The cluster analysis was conducted by using NTSYS software (V 2.10) with unweighted pair-group method, and the clustering chart was plotted (FIGS. 2A-2D). From the clustering chart, it is basically in line with the characteristics of geographical and resource names, and resources with similar or identical germplasm names have very close genetic relationships, proving from another side that the results of this analysis are reliable.

### Example 3 Verification of the Identification of Artemia Resources Using Artemia Core SNP Markers

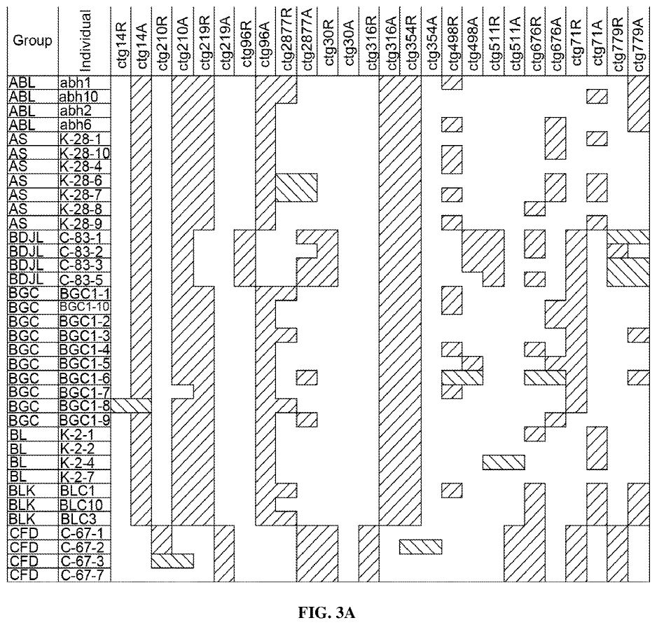

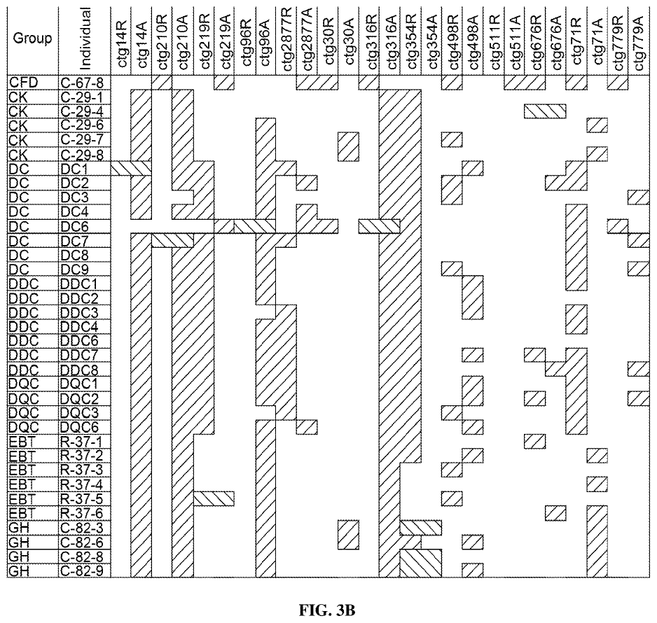

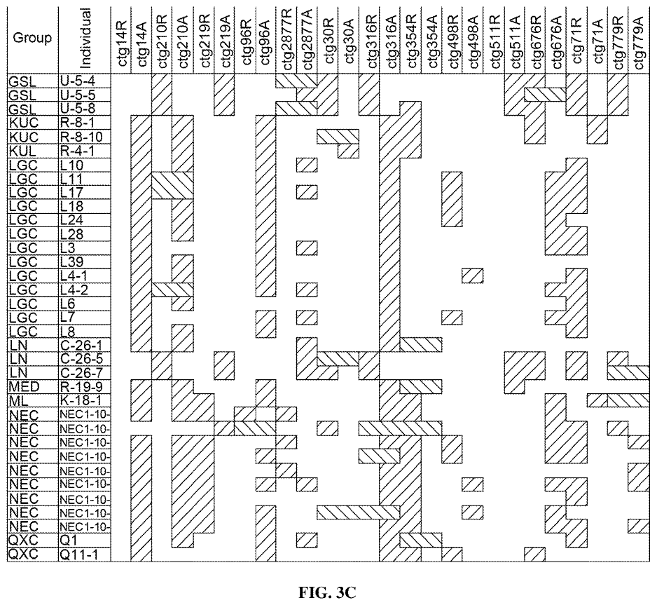

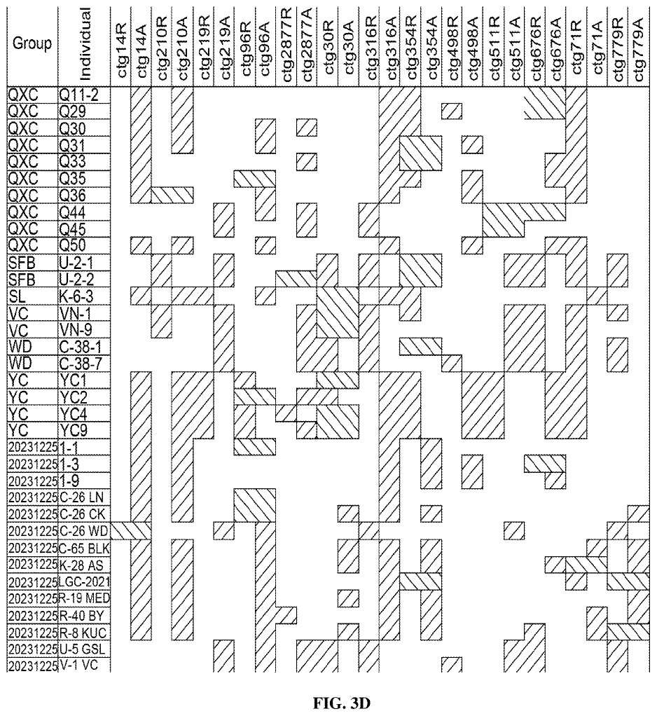

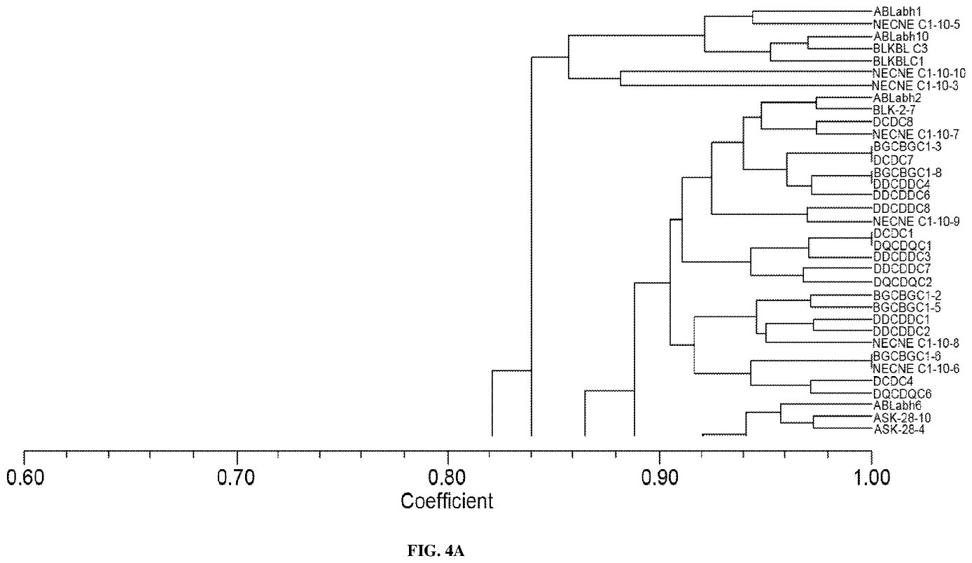

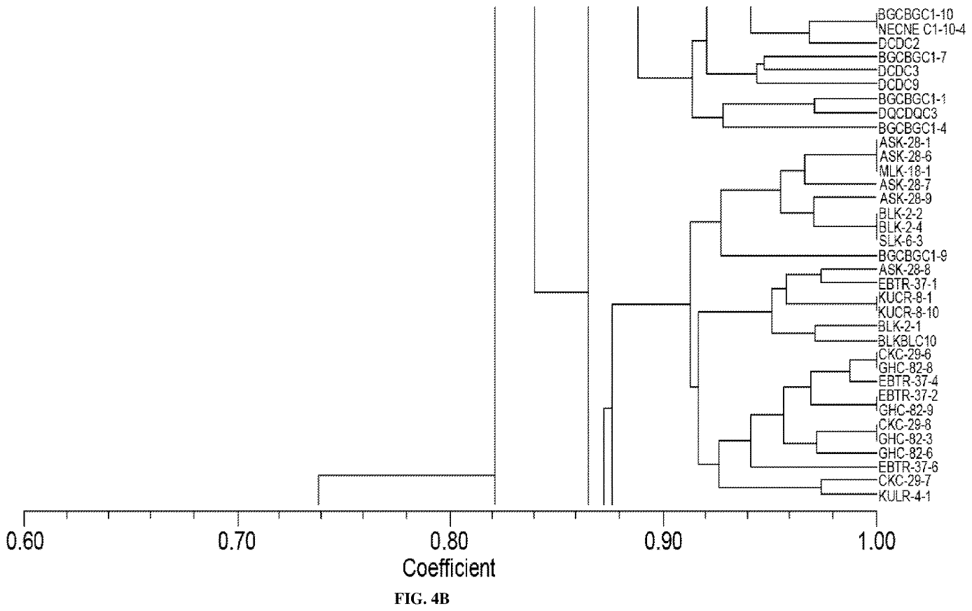

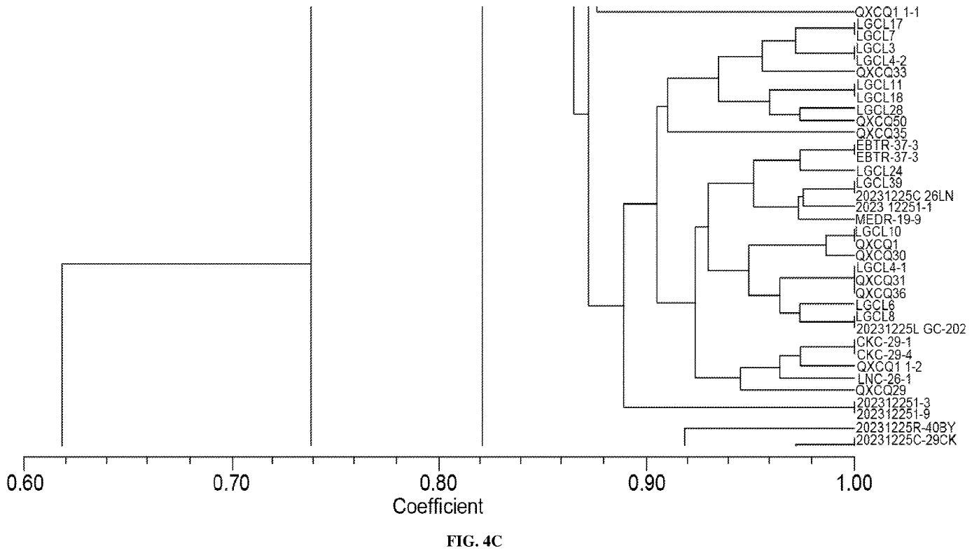

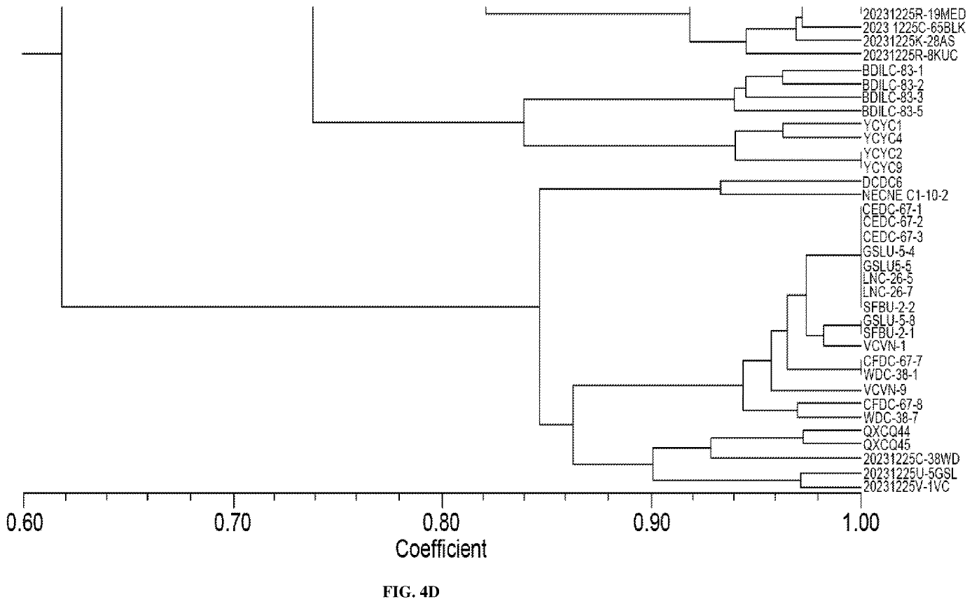

Sixteen germplasms were randomly selected from 290 germplasms. The genetic relationships of the above selected resources were identified using the 13 Artemia core SNP markers and the KASP primers provided by the present disclosure. The specific experimental steps were as follows:
(1) Preparation of DNA template: Artemia eggs were selected from 16 germplasms. Genomic DNAs were extracted by using assay kit method.
(2) Design and synthesis of KASP primers: when designing KASP primers, the selected SNP loci required the addition of FAM-tagged sequence 5′-GAGGTGACCAAGTTCATGCT-3′ (SEQ ID NO: 40) at the 5′-end of the upstream primer-Primer_AlleleX, and HEX-tagged sequence 5′-GAGGTCGGAGTCAACGGATT-3′ (SEQ ID NO: 41) at the 5′-end of the upstream primer-Primer_AlleleY. The primers were synthesized by Beijing Liuhe BGI Co., Ltd. Details of primers were shown in Table 2.
(3) The reaction system was shown in Table 3.
(4) The reaction conditions were as follows: pre-denaturation at 94° C. for 15 min; the first amplification step consisting of 10 cycles of denaturation at 94° C. for 20 s and extension at 61-55° C. for 60 s; and the second amplification step consisting of 26 cycles of denaturation at 94° C. for 20 s and extension at 55° C. for 60 s.
(5) Result analysis: Data pre-processing and analysis were conducted by karaken software. Genotyping data were exported in the form of Excel files. The bands with the same genotype as the reference genome were marked as ‘1’, those with different genotypes were marked as ‘0’, and the deletions were marked as ‘-’. The SNP fingerprints of 16 samples were constructed by using 13 high-quality KASP markers. After eliminating samples with consistent genotypes, the specific fingerprints of 141 samples were finally obtained (FIGS. 3A-3D). The sequence matrix was constructed. The cluster analysis was conducted by using NTSYS software (V 2.10) with unweighted pair-group method, and the clustering chart was plotted (FIGS. 4A-4D). From the fingerprints and clustering chart, 13 out of the 16 germplasms were identified.

What is claimed is:

1. A chip, containing a set of kompetitive allele-specific PCR (KASP) primers, wherein the set of KASP primers are KASP primers for amplifying an Artemia core molecular marker combination, the Artemia core molecular marker combination is KASP-based Artemia core molecular marker combination consisting of 13 single nucleotide polymorphism (SNP) molecular markers; wherein the 13 SNP molecular markers are Artemia SNP01, Artemia SNP02, Artemia SNP03, Artemia SNP04, Artemia SNP05, Artemia SNP06, Artemia SNP07, Artemia SNP08, Artemia SNP09, Artemia SNP10, Artemia SNP11, Artemia SNP12, and Artemia SNP13, respectively; and
the Artemia SNP01 is located on contig14 of Artemia, at a physical position of 585775, with C as a reference base, and A as a substitution base;
the Artemia SNP02 is located on contig30 of Artemia, at a physical position of 3557862, with T as a reference base, and C as a substitution base;
the Artemia SNP03 is located on contig71 of Artemia, at a physical position of 1303074, with A as a reference base, and T as a substitution base;
the Artemia SNP04 is located on contig96 of Artemia, at a physical position of 4160684, with C as a reference base, and G as a substitution base;
the Artemia SNP05 is located on contig210 of Artemia, at a physical position of 3460211, with G as a reference base, and A as a substitution base;
the Artemia SNP06 is located on contig316 of Artemia, at a physical position of 451626, with T as a reference base, and C as a substitution base;
the Artemia SNP07 is located on contig354 of Artemia, at a physical position of 393026, with A as a reference base, and G as a substitution base;
the Artemia SNP08 is located on contig498 of Artemia, at a physical position of 463045, with C as a reference base, and T as a substitution base;
the Artemia SNP09 is located on contig511 of Artemia, at a physical position of 1564272, with A as a reference base, and G as a substitution base;
the Artemia SNP10 is located on contig676 of Artemia, at a physical position of 177489, with C as a reference base, and T as a substitution base;
the Artemia SNP11 is located on contig779 of Artemia, at a physical position of 1201204, with C as a reference base, and T as a substitution base;
the Artemia SNP12 is located on contig2191 of Artemia, at a physical position of 107679, with A as a reference base, and G as a substitution base;
the Artemia SNP13 is located on contig2877 of Artemia, at a physical position of 71092, with C as a reference base, and T as a substitution base; and
the physical positions of the 13 SNP molecular markers are determined by using a contig sequence mounted on a chromosome of Artemia parthenogenetica as a reference genome; and
wherein the nucleotide sequences of KASP primers for amplifying the Artemia SNP01 are set forth in SEQ ID NOs: 1 to 3;
the nucleotide sequences of KASP primers for amplifying the Artemia SNP02 are set forth in SEQ ID NOs: 4 to 6;
the nucleotide sequences of KASP primers for amplifying the Artemia SNP03 are set forth in SEQ ID NOs: 7 to 9;
the nucleotide sequences of KASP primers for amplifying the Artemia SNP04 are set forth in SEQ ID NOs: 10 to 12;
the nucleotide sequences of KASP primers for amplifying the Artemia SNP05 are set forth in SEQ ID NOs: 13 to 15;
the nucleotide sequences of KASP primers for amplifying the Artemia SNP06 are set forth in SEQ ID NOs: 16 to 18;
the nucleotide sequences of KASP primers for amplifying the Artemia SNP07 are set forth in SEQ ID NOs: 19 to 21;
the nucleotide sequences of KASP primers for amplifying the Artemia SNP08 are set forth in SEQ ID NOs: 22 to 24;
the nucleotide sequences of KASP primers for amplifying the Artemia SNP09 are set forth in SEQ ID NOs: 25 to 27;
the nucleotide sequences of KASP primers for amplifying the Artemia SNP010 are set forth in SEQ ID NOs: 28 to 30;
the nucleotide sequences of KASP primers for amplifying the Artemia SNP011 are set forth in SEQ ID NOs: 31 to 33;
the nucleotide sequences of KASP primers for amplifying the Artemia SNP012 are set forth in SEQ ID NOs: 34 to 36; and
the nucleotide sequences of KASP primers for amplifying the Artemia SNP013 are set forth in SEQ ID NOs: 37 to 39.

2. An assay kit, comprising the KASP primers according to claim 1.

3. A method for constructing an Artemia SNP fingerprint, wherein comprising the steps of using the Artemia core molecular marker combination according to claim 1.

4. A method for constructing an Artemia SNP fingerprint, wherein comprising the steps of using the assay kit according to claim 2.

5. A method for classification of Artemia groups, wherein comprising the steps of using the Artemia core molecular marker combination according to claim 1.

6. A method for classification of Artemia groups, wherein comprising the steps of using the assay kit according to claim 2.

7. A method for detecting an Artemia resource, comprising the following steps:
(1) extracting genomic DNA of an Artemia to be tested, and detecting the genomic DNA by using the Artemia core molecular marker combination according to claim 1, to obtain genotype data of the Artemia to be tested; and
(2) aligning the genotype data of the Artemia to be tested with genotype data of existing Artemia, wherein the Artemia to be tested is identified as a new resource when there are 3 or more differential loci.
